(19) United States
(12) Patent Application Publication
(10) Pub. No.: US 2005/0112718 A1
(43) Pub. Date: May 26, 2005

(54) Culture medium for detecting enterococcus

(75) Inventors: Tomota Nakano, Kanagawa (JP); Ryoichi Kubo, Tokyo (JP)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Kanto Kagaku Kabushiki Kaisha, Tokyo (JP)

(21) Appl. No.: 10/976,065

(22) Filed: Oct. 28, 2004

(30) Foreign Application Priority Data
Oct. 30, 2003 (JP) 2003-370536

(51) Int. Cl. C12Q 1/16; C12N 1/20

(52) U.S. Cl. 435/35; 435/253.6

(57) ABSTRACT

A culture medium is provided that includes a reducing chromogenic reagent that does not give a coloration with Enterococcus faecium. There is also provided a method for differentiating Enterococcus faecium from Enterococcus faecalis in a sample, the method including a step of inoculating the above culture medium with the sample; and a step of differentiating between the two bacterial strains by observing the colony color during culture or after culturing.

### BACKGROUND OF THE INVENTION

1. Technical Field to which the Invention Pertains

The present invention relates to an enterococcus isolation medium and, in particular, to an isolation medium for differentiating, on the isolation medium, Enterococcus faecium (hereinafter abbreviated to E.faecium) from Enterococcus faecalis (hereinafter abbreviated to E.faecalis), which are potential disease-causing bacteria, and/or a method for differentiating enterococci using same.

2. Background Art

Enterococci bacteria are indigenous in the human intestinal tract, oral cavity, etc. and 19 bacterial strains have so far been found. Although these bacteria are very weakly pathogenic, they might in some cases cause endocarditis, urinary tract infection, septicemia, etc. as opportunistic infections. Among enterococci isolated from clinical material such as feces or urine, 80% to 90% thereof is E.faecalis, and the majority of the rest is E.faecium (website of the Ministry of Health, Labor, and Welfare of Japan, <URL:http://icnet.umin.ac.jp/other/vre/htm>). Because of this, these two bacterial strains are considered to be clinically important.

E.faecalis and E.faecium have different susceptibilities to antibiotics and, in particular, about 70% of E.faecium clinical isolates have resistance to penicillins. Because of this, treatment methods for the two bacterial strains are different and differentiation of the two bacterial strains should be carried out at an early stage of testing.

Recently, enterococci having resistance to vancomycin (vancomycin-resistant enterococci, hereinafter abbreviated to VRE) have been found. Although VRE exhibit resistance to vancomycin, there is no difference with respect to other properties such as pathogenicity and antibiotic resistance. With regard to these VRE, E.faecalis and E.faecium account for most of the cases detected, and it is important to differentiate the two bacterial strains.

With regard to a method for detecting enterococci, there are methods in which 5-bromo-4-chloro-3-indolyl-β-D-glucopyranoside, esculin, etc. are added individually to culture medium components (website of Merck KgaA, <URL:http://service.merck.de/microbiology/>), and these are commercially available mainly as culture media for food. However, in these methods all of the enterococci colonies and their surroundings change to a single blue or black to dark gray color, and differentiation of the bacterial strains is therefore impossible.

With regard to a method for detecting VRE, with the object of differentiating the two bacterial strains E.faecalis and E.faecium, a culture medium containing 2,3,5-triphenyltetrazolium chloride in which the two bacterial strains are differentiated by the color of colonies is commercially available for clinical use (JP, A, 2003-70495, website of Nippon Becton Dickinson Company, Ltd., <URL:http://www.bdj.co.jp/press/3tqe0k000000mrlm.html>). However, this culture medium has the problem that, since E.faecalis shows a dark red color reaction, and E.faecium shows a pink color reaction, which are similar colors, it is difficult to differentiate the two bacterial strains.

As hereinbefore described, although early and reliable differentiation and determination are desired, particularly in a clinical environment, the conventional methods and culture media cannot reliably differentiate the two bacterial strains E.faecalis and E.faecium, and the object of carrying out differentiation simply and quickly is not fully satisfied.

### SUMMARY OF THE INVENTION

An object of the present invention is therefore to provide a culture medium and/or a method for conveniently separating and detecting enterococci on the culture medium and conveniently and reliably differentiating E.faecium from E.faecalis.

During an intensive investigation by the present inventors in order to solve the above-mentioned problems, it has surprisingly been found that reliable differentiation of E.faecalis from E.faecium is possible by use of a culture medium in which the two bacterial strains show different color reactions, and as a result of further investigation the present invention has been accomplished. It has also been found that when, in addition to a reducing chromogenic reagent, a β-glucosidase chromogenic substrate is contained in a culture medium and culturing is carried out in the culture medium, enterococci can be detected by the color of colonies in the single culture medium, and this culture medium is suitable for the easy detection and differentiation of E.faecalis from E.faecium.

That is, the present invention relates to a culture medium comprising a reducing chromogenic reagent which does not give a coloration with E.faecium.

Furthermore, the present invention relates to the culture medium wherein the reducing chromogenic reagent is 2,3,5-triphenyltetrazolium chloride.

Moreover, the present invention relates to the culture medium wherein it further comprises peptone, glucose, yeast extract, and agar.

Furthermore, the present invention relates to the culture medium wherein it comprises 4 to 6 g/L of peptone, 0.2 to 2 g/L of glucose, 1 to 4 g/L of yeast extract, and 8 to 18 g/L of agar.

Moreover, the present invention relates to the culture medium wherein it further comprises a β-glucosidase chromogenic substrate.

Furthermore, the present invention relates to the culture medium wherein the β-glucosidase chromogenic substrate is 5-bromo-4-chloro-3-indolyl-β-D-glucopyranoside.

Moreover, the present invention relates to the culture medium wherein it further comprises an antibacterial substance against gram negative bacteria.

Furthermore, the present invention relates to the culture medium wherein the antibacterial substance against gram negative bacteria is thallium sulfate.

Moreover, the present invention relates to the culture medium wherein it further comprises vancomycin.

Furthermore, the present invention relates to a method for differentiating Enterococcus faecium from Enterococcus faecalis in a sample, comprising the steps of:
a) inoculating a culture medium with the sample, the culture medium comprising a reducing chromogenic reagent that which not give a coloration with Enterococcus faecium; and
b) differentiating between the two bacterial strains by observing the colony color during culturing or after culturing.

Moreover, the present invention relates to the method wherein the culture medium further comprises a β-glucosidase chromogenic substrate.

A culture medium in which E.faecalis and E.faecium exhibit different degrees of coloration is known in the conventional art (e.g., JP, A, 2003-70495, website of Nippon Becton Dickinson Company, Ltd., <URL:http://www.bdj.co.jp/press/3tqe0k000000mrlm.html>), but there is no known culture medium in which a reducing chromogenic reagent gives a coloration only with E.faecalis, and the reducing chromogenic reagent does not give a coloration with E.faecium. That is, the culture medium of the present invention is the first culture medium that comprises a reducing chromogenic reagent and in which the reducing chromogenic reagent in the culture medium gives a coloration only with E.faecalis colonies, and the reducing chromogenic reagent does not give a coloration with E.faecium.

Furthermore, the method of the present invention comprises inoculating a culture medium comprising a reducing chromogenic reagent with a sample, the reducing chromogenic reagent not giving a coloration with E.faecium, and then observing the colony color after culturing and confirming the presence of E.faecalis when coloration is observed, thus differentiating E.faecalis from E.faecium.

The culture medium of the present invention comprises a reducing chromogenic reagent, and the reducing chromogenic reagent in the culture medium gives a coloration only with E.faecalis, the reducing chromogenic reagent not giving a coloration with E.faecium. In accordance with the culture medium of the present invention, it is therefore possible to isolate and detect enterococci and differentiate E.faecalis from E.faecium.

Furthermore, among the culture media of the present invention, one in which the reducing chromogenic reagent is 2,3,5-triphenyltetrazolium chloride enables the coloration by E.faecalis to be carried out clearly. In accordance with this culture medium, it is therefore possible to more reliably differentiate E.faecalis from E.faecium.

Among the culture media of the present invention, one that further comprises peptone, glucose, yeast extract, and agar enables enterococci to be cultured more efficiently and coloration of the reducing chromogenic reagent by E.faecium to be suppressed. In accordance with this culture medium, it is therefore possible to further reliably differentiate E.faecalis from E.faecium.

Furthermore, among the culture media of the present invention, one that comprises 4 to 6 g/L of peptone, 0.2 to 2 g/L of glucose, 1 to 4 g/L of yeast extract, and 8 to 18 g/L of agar enables enterococci to be cultured yet more efficiently and coloration of the reducing chromogenic reagent by E.faecium to be suppressed. In accordance with this culture medium, it is therefore possible to yet further reliably differentiate E.faecalis from E.faecium.

Moreover, among the culture media of the present invention, one that further comprises a β-glucosidase chromogenic substrate enables E.faecium and E.faecalis in a sample to be detected more rapidly. In accordance with this culture medium, it is therefore possible to differentiate E.faecalis from E.faecium more efficiently.

Furthermore, among the culture media of the present invention, one in which the β-glucosidase chromogenic substrate is 5-bromo-4-chloro-3-indolyl-β-D-glucopyranoside enables coloration by E.faecium to be carried out more clearly. In accordance with this culture medium, it is therefore possible to further reliably differentiate E.faecalis from E.faecium.

Moreover, among the culture media of the present invention, one that comprises an antibacterial substance against gram negative bacteria and, in particular, one that comprises thallium sulfate, enables growth of gram negative bacteria, which are not detection targets, to be suppressed. In accordance with this culture medium, it is therefore possible to further reliably differentiate E.faecalis from E.faecium.

Among the culture media of the present invention, one that further comprises vancomycin enables growth of vancomycin-susceptible enterococci to be suppressed and functions as a VRE-detecting culture medium. In accordance with this culture medium, it is therefore possible to more reliably differentiate vancomycin resistant E.faecalis from vancomycin resistant E.faecium.

As hereinbefore described, since the culture medium for detecting enterococci of the present invention comprises the reducing chromogenic reagent or both the β-glucosidase chromogenic substrate and the reducing chromogenic reagent, it is possible to detect enterococci by the color of colony, and colonies of E.faecium and E.faecalis exhibit different colors in a VRE detection culture medium, which is the above-mentioned vancomycin-containing culture medium. In accordance with the culture media of the present invention, it is therefore possible to differentiate E.faecium from E.faecalis simply and clearly, and provide appropriate medical information to a clinician at an early stage with the differentiation result obtained using the culture media.

In the method of the present invention, after inoculating a culture medium with a sample, the culture medium comprising a reducing chromogenic reagent that does not give a coloration with E.faecium, the colony color during culture or after culturing is observed, and the presence of E.faecalis is confirmed when coloration is observed. In accordance with the method of the present invention, it is therefore possible to conveniently isolate and detect enterococci on a culture medium, and simply and reliably differentiate E.faecalis from E.faecium.

Among the methods of the present invention, one in which the culture medium further comprises a β-glucosidase chromogenic substrate enables E.faecalis and E.faecium in a sample to be detected rapidly. In accordance with this method, it is therefore possible to differentiate E.faecalis from E.faecium more efficiently.

### MODES FOR CARRYING OUT THE INVENTION

The culture medium for detecting enterococci used in the present invention is a culture medium comprising a reducing chromogenic reagent that does not give a coloration with E.faecium. That is, the reducing chromogenic reagent does not change accompanying the growth of E.faecium, but gives a coloration by being metabolized accompanying the growth of E.faecalis. For example, a tetrazolium salt such as 2,3,5-triphenyltetrazolium chloride (TTC), 3-(4,5-dimethyl-2-thiazolyl)-2,5-diphenyl-2H-tetrazolium bromide (MTT), or 3,3-(3,3-dimethoxy-4,4-biphenylene)bis(2,5-diphenyl-2H-tetrazolium chloride) (tetrazolium blue) can be used.

The concentration of the reducing chromogenic reagent used in the culture medium is preferably in the range of 0.001 g/L to 10 g/L, more preferably in the range of 0.01 g/L to 1 g/L, and most preferably in the range of 0.05 g/L to 0.5 g/L.

Since the culture medium of the present invention in which the reducing chromogenic reagent does not give a coloration with E.faecium is a culture medium having the property that the reducing chromogenic reagent gives a coloration by being metabolized accompanying the growth of E.faecalis but does not give a coloration by being metabolized accompanying the growth of E.faecium, it may be a culture medium in which culture medium components such as a nutrient component and the amounts thereof are preferably set so that the reducing chromogenic reagent does not give a coloration with E.faecium.

The components of the culture medium according to the present invention are preferably a nitrogen source component, a carbon source component, a vitamin component, and a support component.

With regard to the nitrogen source component, oligopeptides such as a peptone and amino acids are preferable.

With regard to the carbon source component, a sugar such as glucose is preferable.

With regard to the vitamin component, a single vitamin and/or a mixture of various types of vitamins, or yeast extract is preferable.

With regard to the support component, agar is preferable, and a metal halide such as sodium chloride, magnesium chloride, or calcium chloride may be added, as necessary, as an osmoregulatory component.

Among the culture media of the present invention, one that further comprises peptone, glucose, yeast extract, and agar is therefore preferable. Furthermore, one comprising 4 to 6 g/L of peptone, 0.2 to 2 g/L of glucose, 1 to 4 g/L of yeast extract, and 8 to 18 g/L of agar is more preferable.

Among the culture media of the present invention, one that further comprises a β-glucosidase chromogenic substrate, that is, a culture medium that comprises a reducing chromogenic reagent and a β-glucosidase chromogenic substrate is preferable.

The concentration of the β-glucosidase chromogenic substrate used in the culture medium is preferably in the range of 0.001 g/L to 1 g/L, more preferably in the range of 0.005 g/L to 0.5 g/L, and most preferably in the range of 0.01 g/L to 0.1 g/L.

The β-glucosidase chromogenic substrate gives a coloration as a result of a chromogen of the β-glucosidase chromogenic substrate being liberated by β-glucosidase generated by enterococci. For example, indolyl-glucopyranosides such as 5-bromo-4-chloro-3-indolyl-β-D-glucopyranoside (X-Glu), 5-bromo-6-chloro-3-indolyl-β-D-glucopyranoside, 6-chloro-3-indolyl-β-D-glucopyranoside, 3-indolyl-β-D-glucopyranoside, and 5-bromo-3-indolyl-p-D-glucopyranoside, o-nitrophenyl-p-D-glucopyranoside, 4-methylumbelliferyl-β-D-glucopyranoside, etc. are used in the culture medium of the present invention.

In the culture medium of the present invention further comprising a β-glucosidase chromogenic substrate, it is preferable for the coloration given by the reducing chromogenic reagent and the coloration given by the β-glucosidase chromogenic substrate to belong to different color families from each other. Specifically, for example, when TTC is used as the reducing chromogenic reagent, the coloration given by the β-glucosidase chromogenic substrate should be a color other than red. In this case it is therefore particularly preferable to use, as the β-glucosidase chromogenic substrate, X-Glu, which gives a blue coloration that is clearly visible against the red coloration.

Among the culture media of the present invention, one comprising vancomycin or teicoplanin which, like vancomycin, is a glycopeptide antibacterial agent, is preferable since it can be used as a VRE detection culture medium. One comprising vancomycin is particularly preferable.

The concentration of the glycopeptide antibacterial agent used is preferably in the range of 1 mg/L to 256 mg/L, more preferably in the range of 4 mg/L to 128 mg/L, and most preferably in the range of 6 mg/L to 64 mg/L.

Among the culture media of the present invention, one comprising a gram-negative bacterial growth inhibitor is preferable since the growth of gram negative bacteria in a sample is inhibited. Examples of the gram-negative bacterial growth inhibitors used include polymyxin B, aztreonam, sodium azide, Tween 80, and thallium sulfate, and thallium sulfate is preferable.

The concentration of the gram-negative bacterial growth inhibitor used in the culture medium is preferably in the range of 0.1 g/L to 0.6 g/L, more preferably in the range of 0.2 g/L to 0.5 g/L, and most preferably in the range of 0.3 g/L to 0.4 g/L. These substances may be used singly or as a mixture of several types.

The pH of the culture medium is preferably 6.5 to 7.5, at which enterococci grow effectively, and more preferably 6.8 to 7.2.

The culture medium of the present invention is used as follows.

Firstly, the culture medium is inoculated with a sample, and it is cultured at 30° C. to 45° C. for 12 to 48 hours. After culturing or during culture, properties such as coloration or fluorescence of colonies are inspected. Enterococci are detected as colored colonies. In the culture medium of the present invention that employs a reducing chromogenic reagent such as TTC, when a colony gives a red coloration, E.faecalis is detected. When there is no coloration, it is clear that there is no E.faecalis present. In this case, as necessary, detection of E.faecium is carried out using a culture medium comprising other components.

In the culture medium of the present invention that comprises a β-glucosidase chromogenic substrate such as X-Glu in addition to the reducing chromogenic reagent such as TTC, when the colony gives a red coloration E.faecalis is detected, and when the coloration is blue, E.faecium is detected. That is, the two bacterial strains are differentiated.

In the culture medium of the present invention further comprising the β-glucosidase chromogenic substrate in addition to the reducing chromogenic reagent, the reducing chromogenic reagent and the β-glucosidase chromogenic substrate may be added to the culture medium in advance, or may be added to the culture medium during or after inoculation with a sample. The order in which these substances are added to the culture medium is not limited. From the viewpoint of convenience, it is preferable to add them to the culture medium in advance.

When the culture medium of the present invention comprising the β-glucosidase chromogenic substrate in addition to the reducing chromogenic reagent is inoculated with E.faecalis, E.faecalis reacts with both the reducing chromogenic reagent and the β-glucosidase chromogenic substrate. In the culture medium of the present invention employing TTC as the reducing chromogenic reagent and X-Glu as the β-glucosidase chromogenic substrate, the resulting colonies are red. However, as time elapses a blue color might exude to the area around the red-colored site in some cases.

Examples of samples applied to the culture medium of the present invention include clinical samples such as urine and plasma, food samples such as meat, and hospital samples such as liquids used for wiping infected door knobs, handrails, walls, etc. in a hospital.

The culture medium of the present invention can be provided in agar, liquid, or powder form, these forms containing all the components, including the reducing chromogenic reagent or both the reducing chromogenic reagent and the β-glucosidase chromogenic substrate. The culture medium can also be provided as a small single-use portion of the above-mentioned form in a container such as a petri dish. It is preferable to provide the culture medium in the container such as a petri dish from the viewpoint of portability and convenience.

It is also possible to provide the culture medium having the reducing chromogenic reagent that is not metabolized by E.faecium, and the reducing chromogenic reagent and the β-glucosidase chromogenic substrate in a single container or separate containers as a kit. In this case, considering portability and convenience, it is preferable for the kit to contain equipment such as a sample inoculating stick or a platinum loop.

The method of the present invention for differentiating E.faecium and E.faecalis in a sample comprises the following steps:
a) inoculating a culture medium with the sample, the culture medium comprising a reducing chromogenic reagent which does not give a coloration with Enterococcus faecium; and
b) differentiating between the two bacterial strains by observing the color of a colony during culture or after culturing.

When a colony gives a coloration, E.faecalis is detected.

When the colony does not give a coloration, it is preferable to include a step of inoculating a culture medium comprising a β-glucosidase chromogenic substrate such as X-Glu with the same sample, and examining the coloration. In this case, for example, when it is confirmed that a blue coloration is observed in the culture medium that comprises the β-glucosidase chromogenic substrate, E.faecium is detected.

Among the methods of the present invention, it is preferable for the culture medium to further comprise a β-glucosidase chromogenic substrate since detection of E.faecalis and detection of E.faecium can be carried out at the same time.

The culture time after inoculation depends on the type of each component in the culture medium, but it is preferably 12 to 48 hours at 30° C. to 45° C. from the viewpoint of efficiency and simple operation.

The present invention is explained in further detail below with reference to examples, but the present invention is not limited to these examples.

### EXAMPLE 1

A culture medium of the present invention having the composition and concentration shown in Table 1 was prepared. A test strain was cultured in a soybean casein digest agar culture medium at 35° C. for 24 hours, and a colony was then sampled and diluted with sterile physiological saline. The culture medium of the present invention was inoculated with the diluted liquid and cultured at 35° C. for 48 hours.

As shown in Table 2, E.faecium formed a blue colony, E.faecalis formed a red colony, and the growth of gram negative bacilli was inhibited. In this way, in accordance with the culture medium of the present invention, E.faecalis and E.faecium are easily detected and differentiated.

In the table, ‘tryptone’ is a product name of a peptone manufactured by OXOID Corp.

TABLE 1
Example of composition of culture medium

| Component name | Concentration |
|---|---|
| Tryptone | 5 (g/L) |
| Glucose | 1 (g/L) |
| Yeast Extract | 2.5 (g/L) |
| Tween 80 | 1 (g/L) |
| Thallium sulfate | 330 (mg/L) |
| TTC | 0.1 (g/L) |
| X-Glu | 0.05 (g/L) |
| Agar | 15 (g/L) |
| pH | 7.0 ± 0.2 |

TABLE 2
Growth results

| | | Growth and |
|---|---|---|
| Class | Name of strain | color of colony |
| Enterococci | E. faecalis ATCC51299 | Red |
| | E. faecalis ATCC29212 | Red |
| | E. faecium ATCC6569 | Blue |
| | E. faecium #1 | Blue |
| Gram negative | E. coli ATCC25922 | No growth |
| bacteria | P. aeruginosa IFO3445 | No growth |

### EXAMPLE 2

A culture medium was prepared by adding 6 mg/L of vancomycin to the culture medium having the composition shown in Table 1. A tester strain was cultured in a soybean casein digest agar culture medium at 35° C. for 24 hours, and a colony was then sampled and diluted with sterile physiological saline. The culture medium of the present invention was inoculated with the diluted liquid and cultured at 35° C. for 48 hours. As shown in Table 3, vancomycin resistant E.faecium formed a blue colony, vancomycin resistant E.faecalis formed a red colony, and the growth of vancomycin susceptible enterococci and gram negative bacilli was inhibited. In this way, in accordance with the culture medium of the present invention, vancomycin resistant E.faecalis and E.faecium are easily detected and differentiated.

TABLE 3
Growth results

| | | Growth and |
|---|---|---|
| Class | Name of strain | color of colony |
| VRE | E. faecalis ATCC51299 | Red |
| | E. faecium #1 | Blue |
| Vancomycin susceptible | E. faecalis ATCC29212 | No growth |
| enterococci | E. faecium ATCC6569 | No growth |
| Gram negative bacteria | E. coli ATCC25922 | No growth |
| | P. aeruginosa IFO3445 | No growth |

### EXAMPLE 3

### Investigation of Culture Medium Composition

A tester strain was cultured in a soybean casein digest agar culture medium at 35° C. for 24 hours, and a colony was then sampled and diluted with sterile physiological saline. Culture media having the compositional concentrations shown in Table 4 were inoculated with the diluted liquid and cultured at 35° C. for 24 to 48 hours. It was found that, as shown in Table 5, in culture medium 1 (SPCA), two strains of E.faecalis both reduced TTC and formed a red colony, but E.faecium did not reduce TTC and formed a white colony, and the bacterial strains could thus be distinguished. On the other hand, in culture media 2, 3, and 4 the two bacterial strains both formed a red colony, and therefore the bacterial strains could not be distinguished.

TABLE 4

| | | Culture | Culture | |
|---|---|---|---|---|
| | Culture | medium 2 | medium 3 | Culture |
| | medium 1 | (Nutrient | (Tryptone | medium 4 |
| | (SPC Agar) | Agar) | Soya Agar) | (BHI Agar) |
| Pancreatin | 5.0 g/L | | 15.0 g/L | |
| digest of casein | | | | |
| Yeast extract | 2.5 g/L | 2.0 g/L | | |
| Beef extract for | | 1.0 g/L | | |
| bacteria | | | | |
| Peptone | | 5.0 g/L | | |
| Soya peptone | | | 5.0 g/L | |
| Proteose peptone | | | | 10.0 g/L |
| Cow brain | | | | 12.5 g/L |
| extract powder | | | | |
| Cow heart | | | | 5.0 g/L |
| extract powder | | | | |
| Glucose | 1.0 g/L | | | 2.0 g/L |
| NaCl | | 5.0 g/L | 5.0 g/L | 5.0 g/L |
| Disodium | | | | 2.5 g/L |
| phosphate | | | | |
| Agar | 15.0 g/L | 15.0 g/L | 15.0 g/L | 10.0 g/L |
| TTC | 0.1 g/L | 0.1 g/L | 0.1 g/L | 0.1 g/L |
| pH | 7.0 ± 0.2 | 7.4 ± 0.2 | 7.3 ± 0.2 | 7.4 ± 0.2 |

TABLE 5

| | E. faecalis | E. faecalis | E. faecium |
|---|---|---|---|
| | ATCC51299 | ATCC29212 | ATCC6569 |
| Culture medium 1 | Red | Red | White |
| Culture medium 2 | Red | Red | Red |
| Culture medium 3 | Red | Red | Red |
| Culture medium 4 | Red | Red | Red |

Subsequently, as shown in Table 6, the growth and the color of colonies of the two bacterial strains were compared by changing the amounts of casein pancreatin digest, yeast extract, and glucose, which are components of culture medium 1.

It was found that, as shown in Table 7, neither bacterial strain grew well when the yeast extract was excluded, and the growth could not be improved even by increasing the amount of pancreatin digest of casein or the amount of glucose when the yeast extract was absent. When culture medium 8 was cultured for 48 hours, E.faecium formed a partially pink colony.

TABLE 6

| | Culture | Culture | Culture | Culture | Culture |
|---|---|---|---|---|---|
| | medium 1 | medium 5 | medium 6 | medium 7 | medium 8 |
| Pancreatin | 5.0 g/L | 5.0 g/L | 10.0 g/L | 5.0 g/L | 10.0 g/L |
| digest of | | | | | |
| casein | | | | | |
| Yeast extract | 2.5 g/L | | | | 5 g/L |
| Glucose | 1.0 g/L | 1.0 g/L | | 2.0 g/L | |
| Sorbitol | | | | | 1.0 g/L |
| Agar | 15.0 g/L | 15.0 g/L | 15.0 g/L | 15.0 g/L | 15.0 g/L |
| TTC | 0.1 g/L | 0.1 g/L | 0.1 g/L | 0.1 g/L | 0.1 g/L |
| pH | 7.0 ± 0.2 | 7.0 ± 0.2 | 7.0 ± 0.2 | 7.0 ± 02 | 7.0 ± 0.2 |

TABLE 7

| | E. faecalis | E. faecalis | E. faecium |
|---|---|---|---|
| | ATCC51299 | ATCC29212 | ATCC6569 |
| Culture medium 1 | Red | Red | White |
| Culture medium 5 | White to pink | Red | No growth |
| Culture medium 6 | No growth | No growth | No growth |
| Culture medium 7 | White to pink | Red | No growth |
| Culture medium 8 | Red | Red | White to pink |

From these results it can be seen that the present invention can most suitably be applied with the composition of culture medium 1.

Industrial Applicability

Since the culture medium of the present invention can be utilized for example in medical facilities where VRE infections are treated, and in laboratories for testing, for example, food that uses an antibacterial agent similar to vancomycin, the culture medium will greatly contribute to the development of related industries.

1. A culture medium comprising a reducing chromogenic reagent which does not give a coloration with Enterococcus faecium.

2. The culture medium according to claim 1, wherein the reducing chromogenic reagent is 2,3,5-triphenyltetrazolium chloride.

3. The culture medium according to claim 1, wherein it further comprises peptone, glucose, yeast extract, and agar.

4. The culture medium according to claim 3, wherein it comprises 4 to 6 g/L of peptone, 0.2 to 2 g/L of glucose, 1 to 4 g/L of yeast extract, and 8 to 18 g/L of agar.

5. The culture medium according to claim 1, wherein it further comprises a β-glucosidase chromogenic substrate.

6. The culture medium according to claim 5, wherein the β-glucosidase chromogenic substrate is 5-bromo-4-chloro-3-indolyl-β-D-glucopyranoside.

7. The culture medium according to claim 1, wherein it further comprises an antibacterial substance against gram negative bacteria.

8. The culture medium according to claim 7, wherein the antibacterial substance against gram negative bacteria is thallium sulfate.

9. The culture medium according to claim 1, wherein it further comprises vancomycin.

10. A method for differentiating Enterococcus faecium from Enterococcus faecalis in a sample, comprising the steps of:
a) inoculating a culture medium with the sample, the culture medium comprising a reducing chromogenic reagent which does not give a coloration with Enterococcus faecium; and
b) differentiating the two bacterial strains by observing the colony color during culturing or after culturing.

11. The method according to claim 10, wherein the culture medium further comprises a β-glucosidase chromogenic substrate.
